SEQUENCE LISTING (4 sequences)

SEQ ID NO: 1 - DNA, 916 bp, Leuconostoc mesenteroides
  source 1..916: mol_type = other DNA
cccttttgggcgttttcccaggcggacacttaatgcgttagcttcggcactaagaggcggaaacctcctaacacctagtgttcatcgtttacggtgtggactaccagggtatctaatcctgtttgctacccacactttcgagcctcaacgtcagttgcagtccagtaagccgccttcgccactggtgttcttccatatatctacgcattccaccgctacacatggagttccacttacctctactgcactcaagttaaccagtttccaatgccattccggagttgagctccgggctttcacatcagacttaataaaccgtctgcgctcgctttacgcccaataaatccggataacgctcgggacatacgtattaccgcggctgctggcacgtatttagccgtccctttctggtatggtaccgtcaaactaaaatcatttcctattctagctgttcttcccatacaacagtgctttacgacccgaaagccttcatcacacacgcggcgttgctccatcaggctttcgcccattgtggaagattccctactgcagcctcccgtaggagtttgggccgtgtctcagtcccaatgtggccgatcagtctctcaactcggctatgcatcattgtcttggtaggcctttaccccaccaactaactaatgcaccgcggatccatctctaggtgacgccgaagcgccttttaactttgtgtcatgcgacactaagttttattcggtattagcatctgtttccaaatgttatccccagccttgaggcaggttgtccacgtgttactcacccgttcgccactcacttgaaaggtgcaagcacctttcgctgtgcgttcgacttgcatgtattaggcacgccgccagcgttcatcctgagccatgatccaaactctaaaagggggggg

SEQ ID NO: 2 - DNA, 962 bp, Leuconostoc mesenteroides
  source 1..962: mol_type = other DNA
ccactcggagtctccaggcggacacttaatgcgttagcttcggcactaagaggcggaaacctcctaacacctagtgttcatcgtttacggtgtggactaccagggtatctaatcctgtttgctacccacactttcgagcctcaacgtcagttgcagtccagtaagccgccttcgccactggtgttcttccatatatctacgcattccaccgctacacatggagttccacttacctctactgcactcaagttaaccagtttccaatgccattccggagttgagctccgggctttcacatcagacttaataaaccgtctgcgctcgctttacgcccaataaatccggataacgctcgggacatacgtattaccgcggctgctggcacgtatttagccgtccctttctggtatggtaccgtcaaactaaaatcatttcctattctagctgttcttcccatacaacagtgctttacgacccgaaagccttcatcacacacgcggcgttgctccatcaggctttcgcccattgtggaagattccctactgcagcctcccgtaggagtttgggccgtgtctcagtcccaatgtggccgatcagtctctcaactcggctatgcatcattgtcttggtaggcctttaccccaccaactaactaatgcaccgcggatccatctctaggtgacgccgaagcgccttttaactttgtgtcatgcgacactaagttttattcggtattagcatctgtttccaaatgttatccccagccttgaggcaggttgtccacgtgttactcacccgttcgccactcacttgaaaggtgcaagcacctttcgctgtgcgttcgacttgcatgtattaggcacgccgccagcgttcatctgacgaagaaaaacacatatatataaaaatgtacggaaaccaggagaacggaattttgggaacaaggtaaaagaaataaaagt

SEQ ID NO: 3 - DNA, 1539 bp, Leuconostoc mesenteroides
  source 1..1539: mol_type = other DNA
tagagtttgatcatggctcaggatgaacgctggcggcgtgcctaatacatgcaagtcgaacgcacagcgaaaggtgcttgcacctttcaagtgagtggcgaacgggtgagtaacacgtggacaacctgcctcaaggctggggataacatttggaaacagatgctaataccgaataaaacttagtgtcgcatgacacaaagttaaaaggcgcttcggcgtcacctagagatggatccgcggtgcattagttagttggtggggtaaaggcctaccaagacaatgatgcatagccgagttgagagactgatcggccacattgggactgagacacggcccaaactcctacgggaggctgcagtagggaatcttccacaatgggcgaaagcctgatggagcaacgccgcgtgtgtgatgaaggctttcgggtcgtaaagcactgttgtatgggaagaacagctagaataggaaatgattttagtttgacggtaccataccagaaagggacggctaaatacgtgccagcagccgcggtaatacgtatgtcccgagcgttatccggatttattgggcgtaaagcgagcgcagacggtttattaagtctgatgtgaaagcccggagctcaactccggaatggcattggaaactggttaacttgagtgcagtagaggtaagtggaactccatgtgtagcggtggaatgcgtagatatatggaagaacaccagtggcgaaggcggcttactggactgcaactgacgttgaggctcgaaagtgtgggtagcaaacaggattagataccctggtagtccacaccgtaaacgatgaacactaggtgttaggaggtttccgcctcttagtgccgaagctaacgcattaagtgttccgcctggggagtacgaccgcaaggttgaaactcaaaggaattgacggggacccgcacaagcggtggagcatgtggtttaattcgaagcaacgcgaagaaccttaccaggtcttgacatcctttgaagcttttagagatagaagtgttctcttcggagacaaagtgacaggtggtgcatggtcgtcgtcagctcgtgtcgtgagatgttgggttaagtcccgcaacgagcgcaacccttattgttagttgccagcattcagatgggcactctagcgagactgccggtgacaaaccggaggaaggcggggacgacgtcagatcatcatgccccttatgacctgggctacacacgtgctacaatggcgtatacaacgagttgccaacccgcgagggtgagctaatctcttaaagtacgtctcagttcggattgtagtctgcaactcgactacatgaagtcggaatcgctagtaatcgcggatcagcacgccgcggtgaatacgttcccgggtcttgtacacaccgcccgtcacaccatgggagtttgtaatgcccaaagccggtggcctaaccttttaggaaggagccgtctaaggcaggacagatgactggggtgaagtcgtaacaaggtagccgtaggagaacctgcggttggatcacc

SEQ ID NO: 4 - DNA, 1507 bp, Leuconostoc mesenteroides
  source 1..1507: mol_type = other DNA
gctcaggatgaacgctggcggcgtgcctaatacatgcaagtcgaacgcacagcgaaaggtgcttgcacctttcaagtgagtggcgaacgggtgagtaacacgtggacaacctgcctcaaggctggggataacatttggaaacagatgctaataccgaataaaacttagtgtcgcatgacacaaagttaaaaggcgcttcggcgtcacctagagatggatccgcggtgcattagttagttggtggggtaaaggcctaccaagacaatgatgcatagccgagttgagagactgatcggccacattgggactgagacacggcccaaactcctacgggaggctgcagtagggaatcttccacaatgggcgaaagcctgatggagcaacgccgcgtgtgtgatgaaggctttcgggtcgtaaagcactgttgtatgggaagaacagctagaataggaaatgattttagtttgacggtaccataccagaaagggacggctaaatacgtgccagcagccgcggtaatacgtatgtcccgagcgttatccggatttattgggcgtaaagcgagcgcagacggtttattaagtctgatgtgaaagcccggagctcaactccggaatggcattggaaactggttaacttgagtgcagtagaggtaagtggaactccatgtgtagcggtggaatgcgtagatatatggaagaacaccagtggcgaaggcggcttactggactgcaactgacgttgaggctcgaaagtgtgggtagcaaacaggattagataccctggtagtccacaccgtaaacgatgaacactaggtgttaggaggtttccgcctcttagtgccgaagctaacgcattaagtgttccgcctggggagtacgaccgcaaggttgaaactcaaaggaattgacggggacccgcacaagcggtggagcatgtggtttaattcgaagcaacgcgaagaaccttaccaggtcttgacatcctttgaagcttttagagatagaagtgttctcttcggagacaaagtgacaggtggtgcatggtcgtcgtcagctcgtgtcgtgagatgttgggttaagtcccgcaacgagcgcaacccttattgttagttgccagcattcagatgggcactctagcgagactgccggtgacaaaccggaggaaggcggggacgacgtcagatcatcatgccccttatgacctgggctacacacgtgctacaatggcgtatacaacgagttgccaacccgcgagggtgagctaatctcttaaagtacgtctcagttcggattgtagtctgcaactcgactacatgaagtcggaatcgctagtaatcgcggatcagcacgccgcggtgaatacgttcccgggtcttgtacacaccgcccgtcacaccatgggagtttgtaatgcccaaagccggtggcctaaccttttaggaaggagccgtctaaggcaggacagatgactggggtgaagtcgtaacaaggtagccgtaggagaa

(19) United States
(12) Patent Application Publication
(10) Pub. No.: US 2026/0283203 A1
(43) Pub. Date: Sep. 24, 2026

(54) LEUCONOSTOC STRAIN, AND KIMCHI PRODUCTION METHOD USING SAME

(71) Applicant: CJ CHEILJEDANG CORPORATION, Seoul (KR)

(72) Inventors: Seung Hye CHOI, Yongin-si (KR); Ji Young OH, Seongnam-si (KR); Dong Yun LEE, Suwon-si (KR)

(73) Assignee: CJ CHEILJEDANG CORPORATION, Seoul (KR)

(21) Appl. No.: 19/087,191

(22) Filed: Mar. 21, 2025

Related U.S. Application Data
(63) Continuation-in-part of application No. 16/317,038, filed on Jul. 14, 2017, now abandoned.
(63) Continuation-in-part of application No. 16/317,070, filed on Jul. 14, 2017, now abandoned.

(30) Foreign Application Priority Data
Jul. 15, 2016 (KR) 10-2016-0090284
Jul. 15, 2016 (KR) 10-2016-0090288

(51) Int. Cl. A23L 19/20; A23L 29/00; C12N 1/20

(52) U.S. Cl. CPC A23L 19/20; A23L 29/065; C12N 1/20

(57) ABSTRACT

The present disclosure relates to a Leuconostoc strain, a fermentation starter composition comprising the same, and a method for preparing kimchi using the strain.

A computer readable xml file, entitled “133660-02-7021-US-01_Sequence_Listing_ST26.xml,” created on or about Mar. 21, 2025, with a file size of 9,040 bytes contains the sequence listing for this application and is hereby incorporated by reference in its entirety.

### BACKGROUND

Kimchi is a food prepared by the fermentative action of microorganisms, in which the microorganisms may act to degrade the components of the food and synthesize new components to improve the nutritive value, preference and storage stability of the food.

Conventional kimchi has problems in that gas (mainly carbon dioxide) is produced during distribution of kimchi to cause package inflation, damage to the package, and leakage from the package, and in that the taste quality of kimchi is reduced due to a strong sour taste resulting from overaging.

To overcome such problems, control methods using various packaging technologies such as a polymer film having high gas permeability (Korean Patent Application Publication No. 10-1999-0078725) or application of a one-way valve (Korean Utility Model Publication No. 20-2013-0002058) were reported. However, these methods have limitations in terms of costs or the like in their actual commercialization, and fail to provide a fundamental solution to changes in quality of kimchi, such as a strong sour taste resulting from overaging.

Under this background, the present inventors have made extensive studies to develop a method capable of reducing gas production in the preparation of kimchi. As a result, the present inventors have found that when kimchi is prepared using a specific Leuconostoc strain, gas production can be decreased while inhibiting an increase in sour taste, thereby completing the present application.

### PRIOR ART DOCUMENTS

### Patent Documents

Patent Document 1: KR 10-1999-0078725 A (1999 Nov. 5);
Patent Document 2: KR 20-2013-0002058 A (2013 Apr. 2);
Patent Document 3: KR 10-1999-0078725 A (1999 Nov. 5);
Patent Document 4: KR 20-2013-0002058 A (2013 Apr. 2).

### SUMMARY

The present application relates to a Leuconostoc strain and a method for preparing kimchi using the same.

### DETAILED DESCRIPTION

It is an object of the present application to provide a Leuconostoc mesenteroides CJLM119 strain (KCTC 13043BP) producing decreased amounts of gas.

Another object of the present application is to provide a fermentation starter composition comprising the Leuconostoc mesenteroides CJLM119 strain.

Still another object of the present application is to provide kimchi comprising the Leuconostoc mesenteroides CJLM119 strain or the fermentation starter composition of the present application.

Yet another object of the present application is to provide a method for preparing kimchi, comprising a step of bringing the Leuconostoc mesenteroides CJLM119 strain or the fermentation starter composition of the present application into contact with a material to be fermented.

It is also an object of the present application to provide a Leuconostoc mesenteroides CJLM627 strain (KCTC 13044BP) producing decreased amounts of gas.

Another object of the present application is to provide a fermentation starter composition comprising the Leuconostoc mesenteroides CJLM627 strain.

Still another object of the present application is to provide kimchi comprising the Leuconostoc mesenteroides CJLM627 strain or the fermentation starter composition of the present application.

Yet another object of the present application is to provide a method for preparing kimchi, comprising a step of bringing the Leuconostoc mesenteroides CJLM627 strain or the fermentation starter composition of the present application into contact with a material to be fermented.

Hereinafter, the present application will be described in detail. Meanwhile, the description of one aspect and embodiment disclosed in the present application may also be applied to other aspects and embodiments with respect to common elements. Moreover, all combinations of various elements disclosed in the present application fall within the scope of the present application. In addition, it does not appear that the scope of the present application is limited by the following detailed description. The present disclosure incorporates U.S. Patent Application Publication Nos. 2019/0297908 and 2019/0223478 by reference in their entirety.

To achieve the objects of the present application, in one aspect, the present application provides a Leuconostoc mesenteroides CJLM119 strain (KCTC 13043BP) producing decreased amounts of gas.

According to other embodiments of the present application, the strain of the present application may be a strain producing decreased amounts of acid. Specifically, the acid in the present application may be lactic acid. Thus, when kimchi is prepared using the strain of the present application, the taste quality of kimchi can be maintained by inhibiting an increase in sour taste, compared to when kimchi is prepared using other Leuconostoc mesenteroides strains.

According to another embodiment of the present application, the strain of the present application may be a strain producing increased amounts of mannitol. Mannitol gives a cooling feeling and a refreshing taste to kimchi, suppresses sour taste, and also inhibit the proliferation of over-acidifying microorganisms to prevent kimchi from being excessively fermented.

According to still another embodiment of the present application, the strain of the present application showed negative results in all gelatin liquefaction, toxic metabolite (e.g., ammonia) production, phenylalanine deaminase production, and hemolysis tests, indicating that it is safe even when it is applied to food.

According to still another embodiment of the present application, the strain of the present application may comprise 16s rRNA comprising a sequence of SEQ ID NO: 1.

The Leuconostoc mesenteroides CJLM119 strain of the present application may comprise a cell wall fragment, living cell or dried cell of the strain.

In another aspect, the present application provides A fermentation starter composition comprising a Leuconostoc mesenteroides CJLM119 strain (KCTC 13043BP) or a culture thereof.

The fermentation starter composition of the present application may comprise the Leuconostoc mesenteroides CJLM119 strain (KCTC 13043BP) of the present application at a concentration of 10⁷ cfu/ml or more, specifically, 10⁷ cfu/ml to 10¹³ cfu/ml, or 10⁹ cfu/ml to 10¹² cfu/ml.

As used herein, the term “culture” means a material resulting from culture of the Leuconostoc mesenteroides CJLM119 strain (KCTC 13043BP) of the present application after inoculation into medium. Specifically, the culture of the present application may include a culture itself obtained by culturing the strain of the present application (that is, the culture may include the Leuconostoc mesenteroides CJLM119 strain (KCTC 13043BP) of the present application, medium or a metabolic product of the strain), a filtrate (e.g., a centrifuged supernatant) obtained by filtering or centrifuging the culture to remove the strain, or the like. In addition, the culture of the present application may include one obtained by drying (e.g., freeze-drying) and powdering the culture.

According to one embodiment of the present application, culturing in the present application may be performed at a temperature of 10° C. to 30° C. for 6 to 48 hours. Specifically, the culturing may be performed at a temperature of 20° C. to 30° C. for 12 to 36 hours or 18 to 30 hours.

The medium that is used in the present application is not limited and may be any known medium for lactic acid bacteria. Specifically, the medium that is used in the present application may include a carbon source and a nitrogen source. More specifically, the carbon source may be one or more selected from the group consisting of sucrose, glucose and fructose, and the nitrogen source may be one or more selected from the group consisting of yeast extract, peptone, beef extract, malt extract, corn steep liquor, ammonium citrate, ammonium sulfate, ammonium chloride, ammonium phosphate, ammonium carbonate, and ammonium nitrate. The medium that is used in the present application may further include one or more selected from the group consisting of Tween 80, sodium citrate, potassium phosphate, sodium acetate, manganese sulfate, magnesium sulfate, and distilled water.

As used herein, the term “fermentation starter” means an agent that is artificially applied to a material to be fermented in order to support the start of fermentation.

According to one embodiment of the present application, the fermentation starter composition may be in a liquid or powder form.

According to other embodiments of the present application, the fermentation starter composition of the present application may further comprise a cryoprotectant. More specifically, the fermentation starter composition may further comprise one or more cryoprotectants selected from the group consisting of glycerol, trehalose, maltodextrin, powdered skim milk, and starch. The cryoprotectant that is used in the present application may be comprised in an amount of 0.01 wt % to 20 wt %, or 0.01 wt % to 10 wt %, based on the total weight of the fermentation starter composition of the present application. Specifically, in the present application, glycerol may be comprised in an amount of 5 to 20 wt % in the fermentation starter composition; trehalose may be comprised in an amount of 2 to 10 wt % in the fermentation starter composition; powdered skim milk may be comprised in an amount of 0.5 to 2 wt % in the fermentation starter composition; and starch may be comprised in an amount of 0.1 to 1 wt % in the fermentation starter composition.

According to another embodiment of the present application, the fermentation starter composition of the present application may further comprise an excipient. Specifically, the excipient that is used in the present application may be one or more selected from the group consisting of glucose, dextrin and powdered skim milk. More specifically, the excipient that is used in the present application may be comprised in an amount of 75 to 95 wt %, or 85 to 95 wt %, based on the total weight of the fermentation starter composition of the present application.

In still another aspect, the present application provides kimchi comprising the Leuconostoc mesenteroides CJLM119 strain (KCTC 13043BP) of the present application or the fermentation starter composition of the present application.

As used herein, the term “kimchi” means a food obtained by salting vegetables (e.g., Chinese cabbage, radish, green onion, leaf mustard and cucumber, etc.) and adding seasonings (e.g., red pepper powder, garlic, ginger and pickled fish, etc.) to the salted vegetables, followed by fermentation.

The kimchi of the present application may further comprise known food-acceptable additives. Specifically, the kimchi of the present application may further comprise natural fragrance such as plum fragrance, lemon fragrance, pine apple fragrance, herb fragrance or the like; natural pigment such as natural fruit juice, chlorophyllin, flavonoid or the like; a sweetening component such as fructose, honey, sugar alcohol or sugar; or an acidulant such as citric acid or sodium citrate.

In still another aspect, the present application provides a method for preparing kimchi, comprising a step of bringing the Leuconostoc mesenteroides CJLM119 strain (KCTC 13043BP) of the present application or the fermentation starter composition of the present application into contact with a material to be fermented.

According to other embodiments of the present application, the Leuconostoc mesenteroides CJLM119 strain (KCTC 13043BP) of the present application or the fermentation starter composition of the present application may be brought into contact with a material to be fermented in an amount of 0.01 to 3 wt %, 0.1 to 3 wt %, 0.5 to 3 wt %, or 0.5 wt % to 2 wt %, based on the total weight of the material to be fermented.

In addition, the contacting in the present application may be performed at a temperature of 3 to 10° C. or 5 to 10° C. for 1 to 90 days, 10 to 90 days, 10 to 60 days, 20 to 60 days, or 20 to 40 days.

In still another aspect, the present application provides kimchi prepared by the method for preparing kimchi according to the present application.

To achieve the objects of the present application, in one aspect, the present application provides a Leuconostoc mesenteroides CJLM627 strain (KCTC 13044BP) producing decreased amounts of gas. The Leuconostoc mesenteroides CJLM627 strain (KCTC 13044BP) of the present application may be Leuconostoc mesenteroides subsp. Dextranicum.

According to other embodiments of the present application, the strain of the present application may be a strain producing decreased amounts of acid. Specifically, the acid in the present application may be lactic acid. Thus, when kimchi is prepared using the strain of the present application, the taste quality of kimchi can be maintained by inhibiting an increase in sour taste, compared to when kimchi is prepared using other Leuconostoc mesenteroides strains.

According to another embodiment of the present application, the strain of the present application may be a strain producing increased amounts of mannitol. Mannitol gives a cooling feeling and a refreshing taste to kimchi, suppresses sour taste, and also inhibit the proliferation of over-acidifying microorganisms to prevent kimchi from being excessively fermented.

According to still another embodiment of the present application, the strain of the present application showed negative results in all gelatin liquefaction, toxic metabolite (e.g., ammonia) production, phenylalanine deaminase production, and hemolysis tests, indicating that it is safe even when it is applied to food.

According to still another embodiment of the present application, the strain of the present application may comprise 16s rRNA comprising a sequence of SEQ ID NO: 2.

The Leuconostoc mesenteroides CJLM627 strain of the present application may comprise a cell wall fragment, living cell or dried cell of the strain.

In another aspect, the present application provides a fermentation starter composition comprising a Leuconostoc mesenteroides CJLM627 strain (KCTC 13044BP) or a culture thereof.

The fermentation starter composition of the present application may comprise the Leuconostoc mesenteroides CJLM627 strain (KCTC 13044BP) of the present application at a concentration of 107 cfu/ml or more, specifically, 107 cfu/ml to 1013 cfu/ml, or 109 cfu/ml to 1012 cfu/ml.

As used herein, the term “culture” means a material resulting from culture of the Leuconostoc mesenteroides CJLM627 strain (KCTC 13044BP) of the present application after inoculation into medium. Specifically, the culture of the present application may include a culture itself obtained by culturing the strain of the present application (that is, the culture may include the Leuconostoc mesenteroides CJLM627 strain (KCTC 13044BP) of the present application, medium or a metabolic product of the strain), a filtrate (e.g., a centrifuged supernatant) obtained by filtering or centrifuging the culture to remove the strain, or the like. In addition, the culture of the present application may include one obtained by drying (e.g., freeze-drying) and powdering the culture.

According to one embodiment of the present application, culturing in the present application may be performed at a temperature of 10° C. to 30° C. for 6 to 48 hours. Specifically, the culturing may be performed at a temperature of 20° C. to 30° C. for 12 to 36 hours or 18 to 30 hours.

The medium that is used in the present application is not limited and may be any known medium for lactic acid bacteria. Specifically, the medium that is used in the present application may include a carbon source and a nitrogen source. More specifically, the carbon source may be one or more selected from the group consisting of sucrose, glucose and fructose, and the nitrogen source may be one or more selected from the group consisting of yeast extract, peptone, beef extract, malt extract, corn steep liquor, ammonium citrate, ammonium sulfate, ammonium chloride, ammonium phosphate, ammonium carbonate, and ammonium nitrate. The medium that is used in the present application may further include one or more selected from the group consisting of Tween 80, sodium citrate, potassium phosphate, sodium acetate, manganese sulfate, magnesium sulfate, and distilled water.

As used herein, the term “fermentation starter” means an agent that is artificially applied to a material to be fermented in order to support the start of fermentation.

According to one embodiment of the present application, the fermentation starter composition may be in a liquid or powder form.

According to other embodiments of the present application, the fermentation starter composition of the present application may further comprise a cryoprotectant. More specifically, the fermentation starter composition may further comprise one or more cryoprotectants selected from the group consisting of glycerol, trehalose, maltodextrin, powdered skim milk, and starch. The cryoprotectant that is used in the present application may be comprised in an amount of 0.01 wt % to 20 wt %, or 0.01 wt % to 10 wt %, based on the total weight of the fermentation starter composition of the present application. Specifically, in the present application, glycerol may be comprised in an amount of 5 to 20 wt % in the fermentation starter composition; trehalose may be comprised in an amount of 2 to 10 wt % in the fermentation starter composition; powdered skim milk may be comprised in an amount of 0.5 to 2 wt % in the fermentation starter composition; and starch may be comprised in an amount of 0.1 to 1 wt % in the fermentation starter composition.

According to another embodiment of the present application, the fermentation starter composition of the present application may further comprise an excipient. Specifically, the excipient that is used in the present application may be one or more selected from the group consisting of glucose, dextrin and powdered skim milk. More specifically, the excipient that is used in the present application may be comprised in an amount of 75 to 95 wt %, or 85 to 95 wt %, based on the total weight of the fermentation starter composition of the present application.

In still another aspect, the present application provides kimchi comprising the Leuconostoc mesenteroides CJLM627 strain (KCTC 13044BP) of the present application or the fermentation starter composition of the present application.

As used herein, the term “kimchi” means a food obtained by salting vegetables (e.g., Chinese cabbage, radish, green onion, leaf mustard and cucumber, etc.) and adding seasonings (e.g., red pepper powder, garlic, ginger and pickled fish, etc.) to the salted vegetables, followed by fermentation.

The kimchi of the present application may further comprise known food-acceptable additives. Specifically, the kimchi of the present application may further comprise natural fragrance such as plum fragrance, lemon fragrance, pine apple fragrance, herb fragrance or the like; natural pigment such as natural fruit juice, chlorophyllin, flavonoid or the like; a sweetening component such as fructose, honey, sugar alcohol or sugar; or an acidulant such as citric acid or sodium citrate.

In still another aspect, the present application provides a method for preparing kimchi, comprising a step of bringing the Leuconostoc mesenteroides CJLM627 strain (KCTC 13044BP) of the present application or the fermentation starter composition of the present application into contact with a material to be fermented.

According to other embodiments of the present application, the Leuconostoc mesenteroides CJLM627 strain (KCTC 13044BP) of the present application or the fermentation starter composition of the present application may be brought into contact with a material to be fermented in an amount of 0.01 to 3 wt %, 0.1 to 3 wt %, 0.5 to 3 wt %, or 0.5 wt % to 2 wt %, based on the total weight of the material to be fermented.

In addition, the contacting in the present application may be performed at a temperature of 3 to 10° C. or 5 to 10° C. for 1 to 90 days, 10 to 90 days, 10 to 60 days, 20 to 60 days, or 20 to 40 days.

In still another aspect, the present application provides kimchi prepared by the method for preparing kimchi according to the present application.

The Leuconostoc mesenteroides CJLM119 strain (KCTC 13043BP) of the present application produces decreased amounts of gas. Thus, when kimchi is prepared using the strain or a culture thereof as a fermentation starter, gas production during distribution of the kimchi can be decreased, and thus problems such as damage to packages by gas production can be prevented from arising, thereby stabilizing the distribution quality of the kimchi. Furthermore, the strain of the present application produces decreased amount of acid, and thus maintains constant acidity. In addition, the strain of the present application has an excellent ability to produce mannitol, and thus is effective in improving the taste quality of kimchi.

The Leuconostoc mesenteroides CJLM627 strain (KCTC 13044BP) of the present application produces decreased amounts of gas. Thus, when kimchi is prepared using the strain or a culture thereof as a fermentation starter, gas production during distribution of the kimchi can be decreased, and thus problems such as damage to packages by gas production can be prevented from arising, thereby stabilizing the distribution quality of the kimchi. Furthermore, the strain of the present application produces decreased amount of acid, and thus maintains constant acidity. In addition, the strain of the present application has an excellent ability to produce mannitol, and thus is effective in improving the taste quality of kimchi.

1-3. (canceled)

4. A method of preparing kimchi, the method comprising
adding a Leuconostoc mesenteroides strain including the nucleotide sequence of SEQ ID NO: 1 or 2 to Chinese cabbage, and
fermenting the Chinese cabbage, thereby producing kimchi,
wherein the kimchi has reduced gas generation compared to kimchi prepared without the Leuconostoc mesenteroides strain, and
the Leuconostoc mesenteroides strain is a Leuconostoc mesenteroides strain deposited with Accession number KCTC 13043BP or a Leuconostoc mesenteroides strain deposited with Accession number KCTC 13044BP.

5. The method according to claim 4, wherein the strain includes the nucleotide sequence of SEQ ID NO: 1.

6. The method according to claim 4, wherein the strain includes the nucleotide sequence of SEQ ID NO: 2.

7. The method according to claim 4, wherein the strain is added as a fermentation starter, and the method further comprises
producing the fermentation starter by inoculating the strain to a medium comprising at least one selected from the group consisting of yeast extract, peptone, beef extract, malt extract, corn steep liquor, ammonium citrate, ammonium sulfate, ammonium chloride, ammonium phosphate, ammonium carbonate, and ammonium nitrate, Tween 80, polyoxyethylene sorbitan monooleate, sodium citrate, potassium phosphate, sodium acetate, manganese sulfate, and magnesium sulfate.

8. The method according to claim 4, wherein the strain is added at a concentration of 10⁷ cfu/ml or more.

9. The method according to claim 4, wherein the strain is added in a dried form.

10. The method according to claim 4, wherein the strain is added as a fermentation starter, and the fermentation starter is in a powder form.

11. The method according to claim 4, wherein the strain is added as a fermentation starter, and the fermentation starter is freeze-dried form.

12. The method according to claim 4, wherein the strain is added as a fermentation starter, and the fermentation starter further comprises one or more cryoprotectants selected from the group consisting of glycerol, trehalose, maltodextrin, powdered skim milk, and starch.

13. The method according to claim 12, wherein the one or more cryoprotectants are present in an amount of 0.01 wt % to 20 wt % based on the total weight of the fermentation starter.

14. The method according to claim 12, wherein the cryoprotectants excludes starch.

15. The method according to claim 4, wherein the Leuconostoc mesenteroides strain is the Leuconostoc mesenteroides strain deposited with Accession number KCTC 13043BP.

16. The method according to claim 4, wherein the Leuconostoc mesenteroides strain is the Leuconostoc mesenteroides strain deposited with Accession number KCTC 13044BP.
